(19) United States
(12) Patent Application Publication
(10) Pub. No.: US 2022/0184130 A1
(43) Pub. Date: Jun. 16, 2022

(54) ARTIFICIAL SIGNALLING MOLECULE

(71) Applicant: Medizinische Hochschule Hannover, Hannover (DE)

(72) Inventors: Rainer Blasczyk, Isernhagen (DE); Britta Eiz-Vesper, Hannover (DE); Constanca Ferreira de Figueiredo, Neustadt am Rübenberge (DE); Anna Dragon, Hannover (DE)

(21) Appl. No.: 17/605,180

(22) PCT Filed: Apr. 30, 2020
(86) PCT No.: PCT/EP2020/062148

§ 371 Date: Oct. 20, 2021

(30) Foreign Application Priority Data
Apr. 30, 2019 (EP) 19172048.1

(51) Int. Cl. A61K 35/17; C07K 14/725; C07K 14/705; C07K 14/74

(52) U.S. Cl. CPC A61K 35/17; C07K 14/7051; C07K 2319/03; C07K 14/70539; C07K 14/70521

(57) ABSTRACT

A signalling molecule and an immune cell expressing the signalling molecule for use in the treatment of an undesired immune activity, which signalling molecule is a fusion protein which comprises a ligand domain, a spacer, a transmembrane domain, and at least one intracellular signalling domain, wherein the ligand domain comprises at least one epitope or all of the epitopes of the cognate antigen, or the cognate antigen, which cognate antigen is the target of the undesired immune activity.

The present invention relates to an artificial signalling molecule, which is a fusion protein, for expression in an immune cell, specifically for use in medical treatment. The artificial signalling molecule and an immune cell expressing the artificial signalling molecule are suitable for use in the treatment of undesired immune responses, e.g. for suppression of undesired immune activity, specifically for use in the treatment of immune rejections against transplants, e.g. treatment of HvG disease, for use in the treatment of autoimmune diseases, or for use in the treatment of allergies.

The artificial signalling molecule is designed to direct the immune cell expressing it against immune cells, e.g. T-cells, B-cells or NK cells, which cause the undesired immune response.

### STATE OF THE ART

For suppression of undesired immune activities, it is generally known to suppress the general activity of the immune system.

WO 2018/001874 describes CAR molecules for expression in Treg cells for use in the treatment of HvG disease in a transplant recipient, wherein the CAR molecules generate a suppressive activity in the vicinity of the transplant.

### OBJECT OF THE INVENTION

It is an object of the invention to provide an alternative for use in the treatment of undesired immune activity, especially for treatment of undesired immune responses which are directed against specific cognate antigens, e.g. for use in the treatment of HvG disease, in the treatment of autoimmune disease, or for treatment of an allergy.

### DESCRIPTION OF THE INVENTION

The invention achieves the object by the features of the claims, especially by providing a signalling molecule and an immune cell expressing the signalling molecule for use in the treatment of an undesired immune activity, which signalling molecule is a fusion protein which comprises or consists of a ligand domain, a spacer, a transmembrane domain, and at least one intracellular signalling domain, wherein the ligand domain comprises at least one epitope or all of the epitopes of the cognate antigen, or the cognate antigen, which cognate antigen is the target of the undesired immune activity. Preferably, especially in heterodimeric ligand domains with the two ligand domains, which are e.g. similar in size, the signalling molecule contains a dimerization domain arranged in its extracellular portion, e.g. arranged between the ligand domain and the transmembrane domain. Preferably, the domains of the signalling molecule, which comprise or consist of the ligand domain, a spacer, a transmembrane domain, and at least one intracellular signalling domain, optionally a dimerization domain between the ligand domain and the transmembrane domain, preferably the extracellular part of a HLA transmembrane domain at the C-terminus of the ligand domain, preferably a linker, e.g. of 8 to 12 amino acids, between the extracellular part of the HLA transmembrane domain and the dimerization domain or the spacer, are linked to one another from N-terminus to C-terminus, more preferably directly linked to one another form N-terminus to C-terminus. The immune cell expressing the signalling molecule contains a nucleic acid sequence encoding the signalling molecule, which nucleic acid sequence preferably is integrated into the cellular genome, e.g. by transduction using a viral vector, or by transfection of an encoding nucleic acid sequence.

The ligand domain accordingly comprises or consists of at least one, preferably all, of the epitopes, of the cognate antigen, e.g. the ligand domain can comprise or consist of the cognate antigen for a T-cell receptor (TCR) and/or for a B-cell receptor (BCR). The undesired immune activity to be treated is directed against the cognate antigen. As the signalling molecule of the invention for its specificity contains at least one epitope of the cognate antigen, against which the undesired immune response is directed, binding of a T-cell or B-cell or a NK cell to the ligand domain of the signalling molecule which is expressed by a CD4+ T-cell or a CD8+ T-cell, results in activating the T-cell effector functions specifically against the T-cell or B-cell or NK cell that is bound to the ligand domain of the signalling molecule. As a result of the binding of a CD4+ T-cell or a CD8+ T-cell via its TCR or of a B-cell via its BCR or of a NK cell via its receptor to the signalling molecule when expressed in a CD4+ T-cell or CD8+ T-cell, the bound T-cell or B-cell or NK cell is inactivated or killed, depending on the functional properties of the signalling T-cell. As the binding of a TCR of a T-cell or of a BCR of a B-cell or a KIR of a NK cell is mediated by the ligand domain of the signalling molecule, the inactivation or elimination of the bound T-cell or bound B-cell or bound NK cell is specific for the cognate antigen targeted by the specific receptor of the T-cell or B-cell or NK cell. Accordingly, herein the signalling molecule is also referred to as chimeric ligand receptor (CLR).

As the ligand domain provides a cognate antigen for a TCR or BCR, the ligand domain is no antibody, e.g. no scFv and no other antibody format.

Accordingly, for use in the treatment of HvG disease, the ligand domain, also referred to as the cognate antigen, can be the antigen against which an immune response against a transplant is directed, e.g. the ligand domain, respectively cognate antigen, is a heterologous, e.g. unmatched, HLA class I or class II molecule of the transplant.

Optionally, for use in the treatment of HvG disease, an immune cell expressing the signalling molecule can be for use in the treatment of a patient prior or following to transplantation, especially when the transplant is HLA-mismatched. Further, the immune cell expressing the signalling molecule can be for use in the treatment of a patient who after receiving a transplant has or has not generated memory B-cells directed against mismatched HLA molecules of the transplant.

For use in the treatment of the undesired immune response against a self-antigen, i.e. for use in the treatment of an autoimmune disease, the ligand domain, respectively cognate antigen, can be the self-antigen against which the autoimmune response is directed.

For use in the treatment of an allergy, the ligand domain, respectively cognate antigen, can be the cognate antigen of the allergy, e.g. a peanut antigen such as Ara h 1 (UniProtKB/Swiss-Prot: P43237.1).

The cognate antigen can e.g. be determined as the target of an antibody, of a B-cell receptor (BCR), of a T-cell receptor (TCR) or of a NK cell receptor (e.g. KIR), which occurs during or is characteristic of the undesired immune response. Preferably, the cognate antigen is pre-determined directly or indirectly from a biopsy, e.g. a blood sample, obtained from the patient. For indirect pre-determination of the cognate antigen, the binding of antibody or T-cells of a biopsy can be analysed. For use in the treatment of HvG disease, the cognate antigen can be determined as the non-matched HLA molecule. The TCR of a T-cell, and respectively the BCR of a B-cell, generally are natural components of the patient who has the undesired immune response.

The at least one intracellular signalling domain is a domain which upon reaction of the antigen domain in T-cells activates T-cell effector functions. The intracellular signalling domain can e.g. be a hCD3ξ domain, preferably a combination of a h4-1BB domain and a hCD3ξ (zeta) domain, or a combination of an intracellular hCD28 signalling domain and a hCD3ξ domain.

The transmembrane domain can e.g. be a transmembrane domain of human CD28 (hCD28) or of CD4 (hCD4).

The immune cell expressing the signalling molecule is a T-cell, which preferably is immunologically compatible with the recipient, e.g. autologous to the recipient. In the alternative, the immune cell expressing the signalling molecule is not immunologically compatible with the recipient, e.g. is not HLA-matched with the recipient, so that following administration of the non-compatible immune cell expressing the signalling molecule this may eventually be eliminated by the immune system of the host when it has not been genetically modified in such a way that allorecognition of this immune cell is prevented by e.g. HLA knock down or knock out. Accordingly, for a T-cell which is not immunologically compatible with the recipient, e.g. is not HLA-matched with the recipient, the T-cell may be genetically modified not to express HLA molecules, e.g. genetically modified for knock down or knock out of expression of HLA molecules.

The immune cell expressing the signalling molecule is e.g. a primary CD4+ T-cell or a primary CD8+ T-cell, NK cell and/or a progenitor cell of one of these or a cytotoxic cell line, e.g. NK-92. Herein, the immune cell expressing the signalling molecule is also referred to as a T-cell, which represents a primary CD4+ T-cell or a primary CD8+ T-cell, an NK cell, and a progenitor cell of one of these, or a cytotoxic cell line.

The use of the signalling molecule, and of T-cells expressing the signalling molecule, in the treatment is that the B-cells and the T-cells and the NK cells of the recipient which specifically bind to the antigen domain of the signalling molecule are eliminated, while leaving unaffected other endogenous immune cells of the recipient. Accordingly, the use of the signalling molecule, and of T-cells expressing the signalling molecule, in medical treatment results in the selective inactivation in case of regulatory T cells or elimination of immune cells in case of cytotoxic T cells depending on their cognate receptor matching the ligand domain of the signalling molecule, without a general suppression of the immune system.

A dimerization domain, e.g. a zipper domain, is preferred for signalling molecules that as their ligand domain comprise or consist of two chains, e.g. an alpha chain and a beta chain, of a HLA class II molecule. For example, a first signalling molecule may comprise or consist of, preferably from N-terminus to C-terminus, an antigen domain, a linker, a dimerization domain (also termed zipper), a spacer, a transmembrane domain, and at least one intracellular signalling domain, wherein the dimerization domain is dimerized with the dimerization domain of a second signalling molecule, and wherein the antigen domains of the first and second signalling molecules may be different, e.g. alpha chain and beta chain of a HLA class II molecule. Therein, the second signalling molecule may comprise or consist of a chain of a HLA class II molecule as its ligand domain, a linker, a dimerization domain, a spacer, a transmembrane domain, and at least one intracellular signalling domain. In another embodiment, the second signalling molecule may comprise or consist of a chain of a HLA class II molecule as its ligand domain, a linker and a dimerization domain and is bound by the dimerized dimerization domains to the transmembrane domain and the at least one intracellular signalling domain of the first signalling molecule.

Preferably, the fusion protein contains dimerization domains in each of its constituent proteins, also referred to as signalling molecules, which dimerization domains do not form homodimers but only form heterodimers. Accordingly, each of the signalling molecules, respectively each protein covalently linked to or comprising one of the chains of a ligand domain comprising two chains, contains a different one of a pair of dimerization domains that forms heterodimers. Preferred dimerization domains to form heterodimers are e.g. selected from E and K peptides of an E/K coiled coil structure domain, N-terminal and complementary C-terminal parts of a split intein, e.g. the 36-residue N-terminal portion and the remaining C-terminal portion of a split intein, 1 knob and 1 hole of complementary knob-into-hole engineered human IgG CH3 domains, e.g. comprising mutations such as T366Y, F405A, T394W and/or Y407T, or any other combination pair of complementary protein-protein interaction domains.

In a preferred embodiment, a fusion protein according to the invention consists of a first signalling molecule and a second signalling molecule, wherein the first signalling molecule from N-terminus to C-terminus consists of a ligand domain, a dimerization domain, a spacer, a transmembrane domain, and an intracellular signalling domain. In the preferred embodiment, the second signalling molecule comprises a dimerization domain, the dimerization domain of the first signalling molecule is dimerized with the dimerization domain of the second signalling molecule, and the ligand domain contains at least one epitope of a cognate antigen, against which an undesired immune response is directed.

Therein, the second signalling molecule preferably consists of a ligand domain, a linker and a dimerization domain. Alternatively, the second signalling molecule may consist of a signalling domain, a linker, a dimerization domain, a spacer, a transmembrane domain and an intracellular signalling domain.

Generally herein the domains of a signal molecule are given from the N-terminus of the signal molecule to the C-terminus of the signal molecule.

Generally, for a HLA class II molecule as the ligand domain, the ligand domain can consist of the alpha 1 domain and the beta 1 domain, and optionally the alpha 2 and the beta 2 domains, of the HLA class II heavy chains. Therein, the ligand domain of the first signalling molecule can comprise or consist of the alpha 1 and optionally alpha 2 domains, and the ligand domain of the second signalling molecule can comprise or consist of the beta 1 and optionally beta 2 domains. Alternatively, the ligand domain of the first signalling molecule can comprise or consist of the beta 1 and optionally beta 2 domains, and the ligand domain of the second signalling molecule can comprise or consist of the alpha 1 and optionally alpha 2 domains.

It was found that the arrangement of the extracellular part of the HLA transmembrane domain (in the sequences examples are referred to as extracellular part of transmembrane domain) at the C-terminal end of the alpha 2 domain or of the beta 2 domain results in a more stable molecule. The extracellular part of the HLA transmembrane domain is preferably encoded by exon 4 of a HLA class II and exon 5 of HLA class I encoding gene.

Optionally, for signalling molecules having the heavy chain (alpha chain) of HLA class I as the ligand domain, a β2-microglobulin may be bound by a dimerization domain, wherein e.g. the β2-microglobulin has a dimerization domain at its N-terminus or at its C-terminus. The signalling molecule may comprise or consist of, preferably from N-terminus to C-terminus, a ligand domain which is the heavy chain of a HLA class I molecule, optionally the extracellular part of the HLA transmembrane domain, a linker, a dimerization domain, a spacer, a transmembrane domain, and at least one intracellular signalling domain, wherein the dimerization domain is dimerized with the dimerization domain of a second molecule, which comprises or consists of β2-microglobulin having a dimerization domain at its N-terminus or at its C-terminus.

Herein, the spacer is also referred to as a hinge domain.

Generally, the ligand domain can be a MHC I molecule comprising an alpha chain and a β2-microglobulin according to European patent application No. 19166923.3, filed on 2 Apr. 2019.

Generally, for HLA class I as ligand domain, the ligand domain can consist of the alpha 1 domain and the alpha 2 domain, and optionally the alpha 3 domain, of a HLA class I molecule.

Optionally, the T-cell expressing the CLR can encode two or more different CLR molecules, each having a different ligand domain, e.g. from different HLA class I or different HLA class II molecules or different HLA class I and HLA class II molecules. For HLA class I as the ligand domains the CLR is consisting of at least one, two or three HLA class I heavy chain domains, e.g. of the alpha 1 domain, the alpha 2 domain, and the alpha 3 domain. The light chain, i.e. the beta-2-microglobulin, can be the naturally expressed light chain or a recombinant beta-2-microgloblin, which could optionally also be covalently bound to one of the heavy chain domains. For HLA class II as the ligand domains, the ligand domain can consist of at least one or two alpha chain domains, e.g. of the alpha 1 domain and alpha 2 domain and of at least one or two beta chain domains, e.g. of the beta 1 domain and the beta 2 domain.

The HLA class I or HLA class II antigens are known, e.g. from https://www.ebi.ac.uk/ipd/imgt/hla/download.html, release 0.3.35 or later, which can be downloaded from ftp://ftp.ebi.ac.uk/pub/databases/ipd/imgt/hla/.

Optionally, the signal molecule which as the ligand domain comprises an alpha chain or heavy chain of a HLA class I molecule, this ligand domain can contain at least one mutation in amino acid position No. 74, 223, 224, 225, 226, 227, 229, and 245, wherein the numbering refers to the mature protein, i.e. without the signal peptide, of the alpha chain or heavy chain of a HLA class I molecule, e.g. at least one mutation of 74L, 223A, 224F, 225D, 226A, 227K, 229A, and 245V, in order to reduce or abolish binding of the CD8 of CD8+ T-cells for reducing or abolishing a cytotoxic activity of CD8+ T-cells against T-cells expressing a CLR containing portion of a HLA class I molecule as its ligand domain.

Optionally, the signal molecule which as the ligand domain comprises an alpha and/or beta chain or heavy chains of a HLA class II molecule, this ligand domain can contain at least one mutation in amino acid position Nos. 88, 90 and 176, wherein the numbering refers to the mature protein, i.e. without the signal peptide, of the alpha chain of a HLA class II molecule, resulting in a weakening or abolishing of the interaction of DR od DQ or DP alpha chain with CD4+ T-cells, and/or a mutation in at least one amino acid position of Nos. 46, 54, 55, 56, 104, 114, 116, 134, 135, 136, 137, 138, 139, 141, 142, 143, 144, 145, 148, 158, 160, and 162, wherein the numbering refers to the mature protein, i.e. without the signal peptide, of the beta chain of a HLA class II molecule, in order to weaken or abolish the interaction of DR or DQ or DP beta chain with CD4+ T-cells.

Optionally, the immune cell expressing the at least one signal molecule can further be genetically manipulated to additionally express at least one of the complement inhibitors hDAF (CD55), CD46, or CD59, or a combination of at least two of these, especially for use in the treatment of HvG disease or for use in the treatment of adverse immune reactions of a HLA-mismatched transplant.

Optionally, the fusion protein can comprise a ligand domain consisting of two chains which are associated to one another, e.g. the chains of an MHC class I molecule or the chains of an MHC class II molecule which are not covalently bound to one another, wherein each of the chains of the ligand domain is covalently linked to a separate dimerization domain, and the dimerization domains are dimerized. Therein, the fusion protein comprises or consists of two proteins that are dimerized at the dimerization domains, one dimerization domain of the fusion protein that consists of the one chain of the ligand domain, a (first) dimerization domain, a spacer, a transmembrane domain and an intracellular signalling domain, and the other dimerization domain of the fusion protein that consists of the other one chain of the ligand domain and a (second) dimerization domain, optionally further a spacer, a transmembrane domain and an intracellular signalling domain.

Accordingly in one embodiment, the fusion protein that comprises a ligand domain comprised of two chains, contains two proteins, one containing the one chain of the ligand domain and the other one containing the other chain of the ligand domain, each of these chains of the ligand domain being covalently linked to an individual dimerization domain, of which proteins only one contains, e.g. is covalently linked to, a spacer, a transmembrane domain, and to an intracellular signalling domain. In this embodiment, the protein containing the other one of the chains of the ligand domain consists of this chain of the ligand domain and a dimerization domain, which is dimerized to the dimerization domain of the other protein. Accordingly, in this embodiment, the fusion protein contains only one spacer covalently linked to only one transmembrane domain, which is covalently linked to the intracellular signalling domain.

In another embodiment, in the fusion protein which contains a ligand domain comprised of two chains, each of these chains is covalently linked to an individual dimerization domain, an individual spacer, an individual transmembrane domain, and an individual intracellular signalling domain. Therein, the spacers, transmembrane domains and intracellular signal domains that are each covalently linked to one of the two chains of the ligand domain can have the same amino acid sequence or a different amino acid sequence each.

The invention is now described by way of examples and with reference to figures, which show in

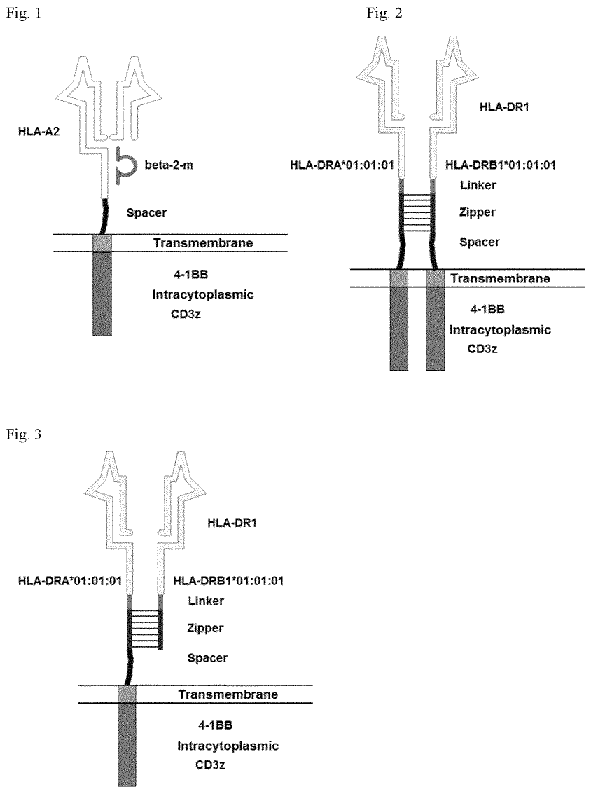

FIG. 1 a schematic drawing of an embodiment of a signalling molecule of the invention,

FIG. 2 a schematic drawing of a further embodiment of a signalling molecule of the invention,

FIG. 3 a schematic drawing of a further embodiment of a signalling molecule of the invention,

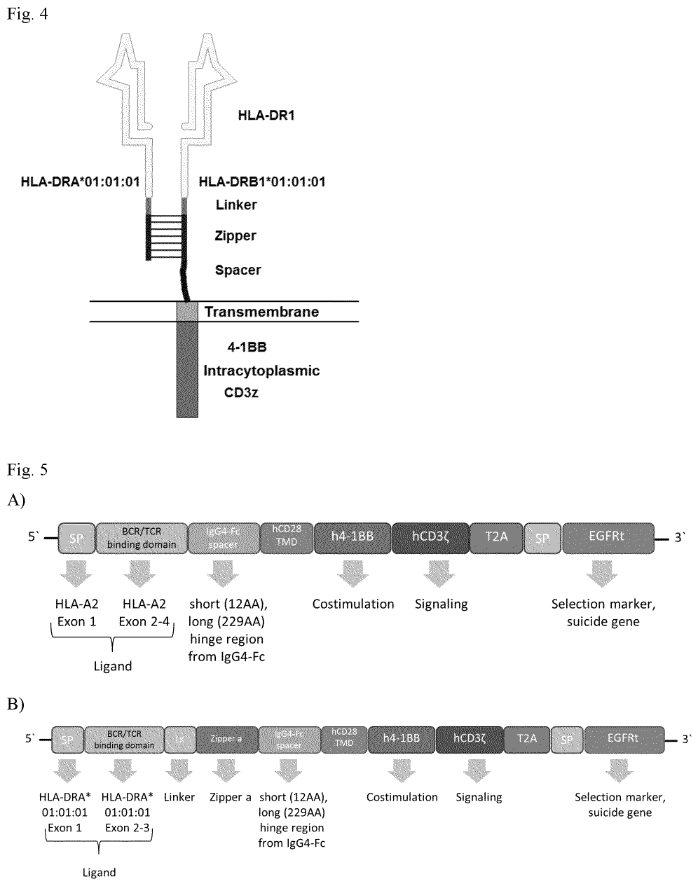

FIG. 4 a schematic drawing of a further embodiment of a signalling molecule of the invention,

FIG. 5 in A), B), C), D) and E) schematic drawings of embodiments of the arrangement of domains of the signalling molecule,

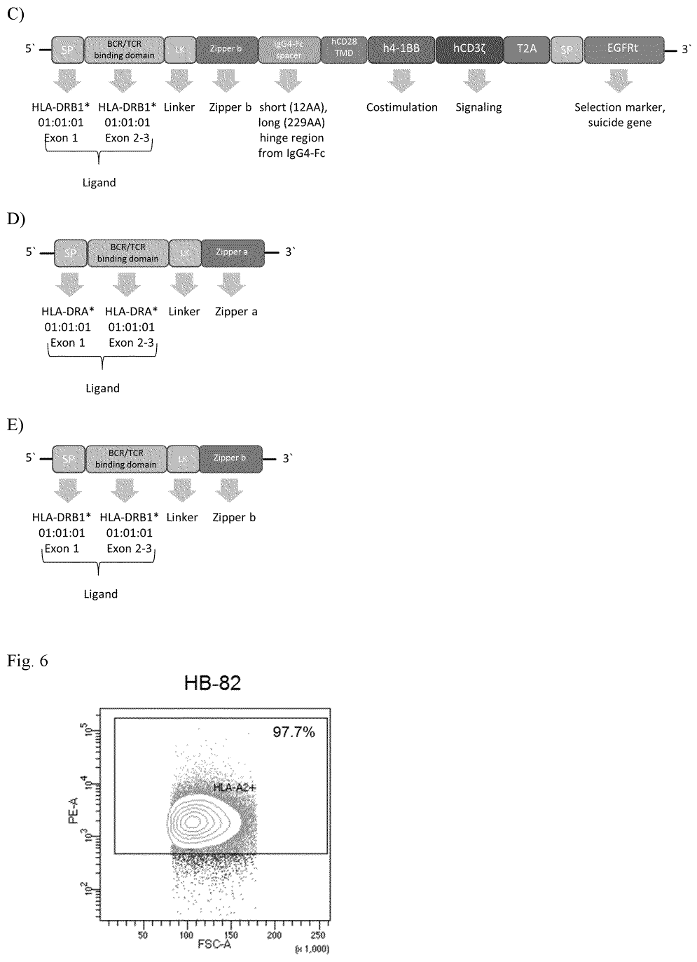

FIG. 6 the FACS result of detecting the CLR when expressed in immune cells by antibody directed against the antigen domain of the CLR,

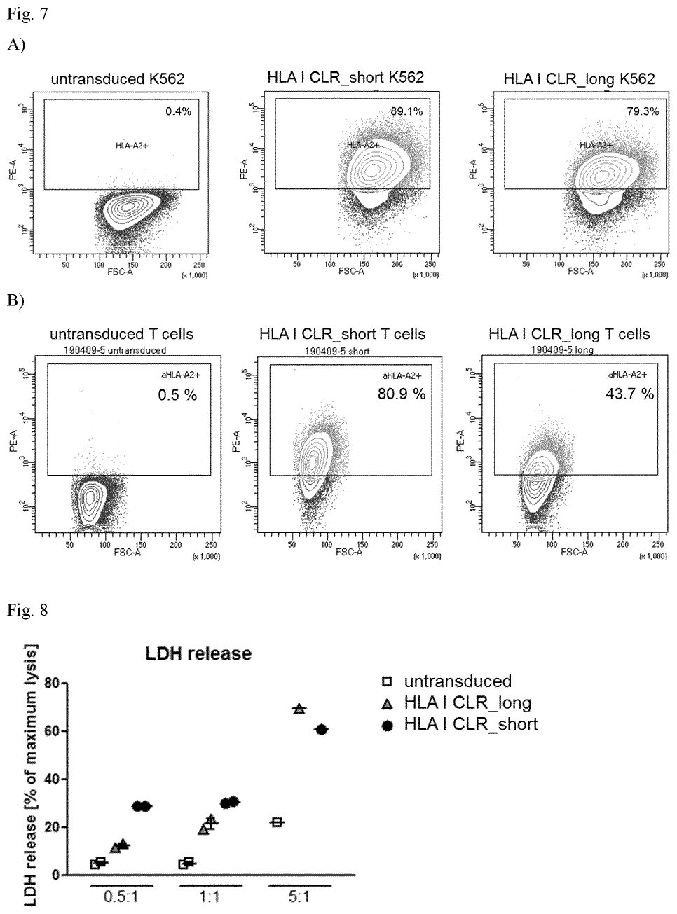

FIG. 7A) the FACS results of detecting the CLR when expressed in control cells (K562) and 7B) when expressed in immune cells (T-cells) by antibody directed against the antigen domain of the CLR,

FIG. 8 LDH release as a measure for cytotoxicity of T-cells expressing the CLR, and

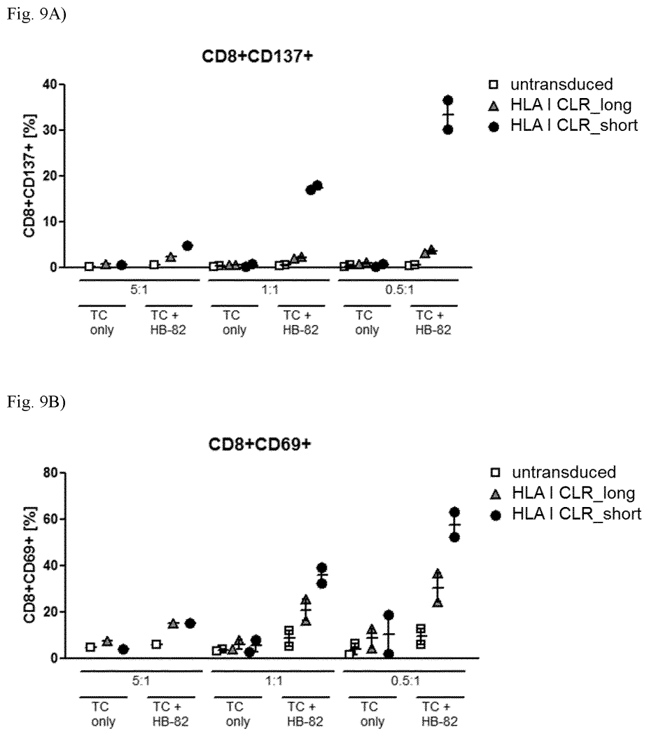

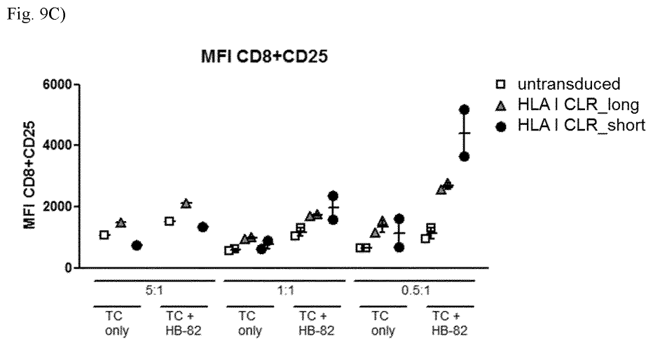

FIG. 9A), B) and C) results of analysis of activation marker expression in T-cells expressing the CLR.

FIG. 1 shows a schematic drawing of a signalling molecule, CLR, of the invention, which is arranged in the membrane of a cellular membrane and consists of an extracellular ligand domain, e.g. a HLA class I molecule, in this example represented by the non-covalently bound light chain beta-2-micrglobulin, a HLA-A*02:01 heavy chain (HLA-A2) consisting of its alpha 1, alpha 2 and alpha 3 domains, and a spacer, a transmembrane domain spanning the cellular membrane, and an intracellularly arranged signalling domain of a combination of the intracellular 4-1BB domain and the CD3ξ (CD3z) domain. This exemplary ligand domain represents the cognate antigen of a HvG disease.

FIG. 2 shows a schematic drawing of a signalling molecule, CLR, of the invention, which comprises two heavy chains and which in a preferred embodiment having an extracellular dimerization domain represented by the two partners of a molecular zipper separated by a linker from each heavy chain and wherein both heavy chains via transmembrane domains are anchored in the membrane of a cellular membrane. This signalling molecule consists of two proteins that are dimerized by their dimerization domains, each containing an extracellular ligand domain, e.g. a HLA class II molecule, in this example represented by a HLA-DR1 antigen consisting of its alpha 1 and alpha 2 domains of the HLA-DR1 alpha chain HLA-DRA*01:01 and the beta 1 and beta 2 domains of the HLA-DR1 beta chain HLA-DRB1*01:01, with each heavy chain linked to a spacer, a transmembrane domain spanning the cellular membrane, and an intracellularly arranged signalling domain of a combination of the intracellular 4-1BB domain and the CD3ξ (CD3z) domain. This exemplary ligand domain represents the cognate antigen of a HvG disease. Generally, for HLA class II as ligand domain, the ligand domain can consist of the alpha 1 domain and the beta 1 domain, and optionally the alpha 2 and the beta 2 domains, of the HLA class II heavy chains. An example for a signalling molecule according to FIG. 2 is given in SEQ ID NO: 10 with a short linker (also referred to as spacer), wherein the zipper a is embodied by the Jun-Zipper. An example for a signalling molecule according to FIG. 2 with a short linker is given in SEQ ID NO: 11, wherein the zipper b is embodied by the Fos-Zipper as zipper b, which can dimerize with the Jun-Zipper of SEQ ID NO: 10.

A further example for a signalling molecule according to FIG. 2, with a long linker, is given in SEQ ID NO: 12, which includes the Jun-Zipper as a dimerizing domain, and in SEQ ID NO: 13 with a Fos-Zipper, for dimerization with the Jun-Zipper of SEQ ID NO: 12.

FIG. 3 shows a schematic drawing of an embodiment of the signalling molecule, CLR, of the invention with the transmembrane and signalling domains of two proteins only attached to one of the two heavy chains, here to the alpha 1 and alpha 2 domains of the HLA-DR1 alpha chain HLA-DRA*01:01. Herein, a first signalling molecule (CLR) consists of the HLA-DR1 alpha chain HLA-DRA*01:01 as its ligand domain, a linker, a dimerization domain (Zipper), a spacer, a transmembrane domain, and a combination of the intracellular 4-1BB domain and the CD3ξ (CD3z) domain as a signalling domain. A second CLR consisting of a ligand domain represented by beta chain of HLA-DR1 (HLA-DRB1*01:01:01), a linker and a dimerization domain (Zipper) is bound to the first CLR by the dimerized dimerization domains. An example for a signalling molecule according to FIG. 3 is given in SEQ ID NO: 14, which contains a short linker and wherein the zipper a is embodied by the Jun-Zipper, for dimerization with the Fos-Zipper of SEQ ID NO: 15.

A further example for a signalling molecule according to FIG. 3, with a long linker, is given in SEQ ID NO: 16, which includes the Jun-Zipper as a dimerizing domain, and in SEQ ID NO: 17 with a Fos-Zipper, e.g. for dimerization with the Jun-Zipper of SEQ ID NO: 12.

FIG. 4 shows a schematic drawing of an embodiment of the signalling molecule, CLR, of the invention with the transmembrane and signalling domains only attached to one of the two heavy chains, here to the beta 1 and beta 2 domains of the HLA-DR1 beta chain. Herein, a first signalling molecule consists of the HLA-DR1 (HLA-DRB1*01:01:01) as its ligand domain, a linker, a dimerization domain (Zipper), a spacer, a transmembrane domain, and a combination of the intracellular 4-1BB domain and the CD3ξ (CD3z) domain as a signalling domain. A second CLR consisting of a ligand domain represented by beta chain of HLA-DRA*01:01:01, a linker and a dimerization domain (Zipper) is bound to the first CLR by the dimerized dimerization domains.

An example for a signalling molecule according to FIG. 4 is given in SEQ ID NO: 18, which contains a short linker and wherein the zipper a is embodied by the Fos-Zipper, for dimerization with the Jun-Zipper of SEQ ID NO: 19, also having a short linker.

A further example for a signalling molecule according to FIG. 4, with a long linker, is given in SEQ ID NO: 20, which includes the Fos-Zipper as a dimerizing domain, and in SEQ ID NO: 21 with a Jun-Zipper, e.g. for dimerization with the Fos-Zipper of SEQ ID NO: 20. Generally, for a HLA class II molecule as ligand domain, the ligand domain can consist of the alpha 1 domain and the beta 1 domain, and optionally the alpha 2 and the beta 2 domain, e.g the alpha 1 and alpha 2 domain or the beta 1 and beta 2 domain, of the HLA class II heavy chains, preferably with the extracellular part of the HLA class II transmembrane domain at the C-terminus of the heavy chains.

FIG. 5 shows preferred signalling molecules of the invention, in FIG. 5A) a nucleic acid sequence encoding, from 5′ to 3′, an embodiment of the signalling molecule and as an optional component, separated by a self-cleaving peptide element derived from Thosea asigna Virus 2A (T2A), a selection marker comprising a secretion signal peptide (SP) and a selection marker and/or suicide gene, represented by truncated epidermal growth factor (EGFRt). The encoded signalling molecule contains, from N-terminus to C-terminus, a ligand domain comprised of a secretion signal peptide (SP, exon 1 of HLA-A*02:01:01) and exons 2-4 of HLA-A*02:01:01, a spacer (IgG4-Fc spacer), a transmembrane domain (hCD28 TMD) of human CD28, and an intracellular signalling domain of a combination of the co-stimulating h4-1BB domain (h4-1BB) and the hCD3ξ domain (hDC3ξ).

The ligand domain acts as the ligand or binding domain for specific binding to a BCR of a B-cell, resp. of a TCR of a T-cell (BCR/TCR binding domain). As generally preferred, the antigen domain can be a portion of or the entire HLA class I heavy chain sequence. In this example, the antigen domain is the truncated HLA-A2 (A*02:01:01) heavy chain consisting of the alpha 1, alpha 2 and alpha 3 domains. An exemplary amino acid sequence is given in SEQ ID NO: 1, consisting of an N-terminal signal peptide and the antigen domain consisting of domains alpha 1, alpha 2 and alpha 3 of HLA-A*02:01.

The spacer can e.g. comprise 10 to 250 amino acids (AA) of a known spacer, e.g. of a Ig hinge region, e.g. 12 AA up to 229 AA of an Ig hinge region, e.g. of the hinge region of IgG4-Fc. Herein, a spacer of 12AA (short) or of 229 AA (long) of the hinge region of IgG4-Fc was used.

In FIG. 5, the preferred extracellular part of a HLA transmembrane domain arranged at the C-terminus of the ligand domain, respectively between the ligand domain and the linker, is not explicitly depicted.

The ligand domain of FIG. 5B) and 5C), which forms the ligand for the TCR, resp. BCR, is comprised of exon 1 of HLA-DRA*01:01:01 (HLA-DRA*01:01:01 Exon 1) and exons 2-3 of HLA-DRA*01:01:01, and exon 1 of HLA-DRB1*01:01:01 and exon 2-3 of HLA-DRB1*01:01:01, respectively. The embodiments comprising portions of or entire HLA class II molecules as ligand domains preferably contain a dimerization domain in order to provide for assembly with the HLA class II beta chain of the HLA class II molecule, also referred to as zipping effect.

FIG. 5B) shows an embodiment, in which a dimerization domain is arranged between the ligand domain and the spacer, wherein as preferred the dimerization domain at its N-terminus comprises a linker (Linker). The dimerization domain is shown as one partner of a molecular or leucine zipper (Zipper a) which can be one of the dimerization partners of e.g. a jun/fos zipper, a basic/acidic zipper or a EE1234L/RR1234L zipper. SEQ ID NO: 2 gives the amino acid sequence of a signal peptide, an antigen domain of domains alpha 1 and alpha 2 of HLA-DRA*01:01, an extracellular part of a transmembrane domain, a linker and Jun zipper (Zipper a) as a dimerization domain.

FIG. 5C) shows an embodiment, in which the dimerization domain is the counterpart of zipper a (Zipper b). SEQ ID NO: 3 gives the amino acid sequence of a signal peptide, a ligand domain of domains beta 1 and beta 2 of HLA-DRB1*01:01, an extracellular part of a transmembrane domain, a linker and Fos zipper (Zipper b) as a dimerization domain.

Alternative amino acid sequences for a linker domain and a dimerization domain are given in SEQ ID NO: 4 and SEQ ID NO: 5, the zipper portions of which can dimerize, and SEQ ID NO: 6 and SEQ ID NO: 7, the zipper portions of which can dimerize.

### EXAMPLE

### Specific Elimination of B-Cells

As an example for immune cells causing an undesired immune activity to be treated, the mouse hybridoma B lymphocyte HB-82 cells were used, which express the HLA-A2 specific antibody BB7.2 as a BCR on their cell surface and in culture secrete the HLA-A2 specific antibody BB7.2 into the supernatant. FIG. 6 shows a FACS result of detecting the BB7.2 antibody on the surface of HB-82 cells stained with anti-mouse IgG (F(ab)2), labelled with Phycoerythrin. This shows that BB7.2 is not only secreted in the supernatant but also strongly expressed on the surface of HB-82 cells.

The antibody produced in the supernatant of HB-82 cells was capable of immunologically staining HLA-A2+ lymphocytes in FACS analysis using as a second antibody anti-mouse IgG (F(ab)₂), labelled with Phycoerythrin as well as killing HLA-A2+ lymphocytes in complement dependent cytotoxicity assays. This shows that the BB7.2 antibody binds to the HLA-A2+ lymphocytes.

In a separate culture, CD8+ T-cells were stimulated by anti-CD3 and anti-CD38 antibodies for controlled expansion. As a control, K562 cells were treated in the same manner. On the next day, the cells were lentivirally transduced with a nucleic acid sequence encoding a CLR according to FIG. 5A), comprising the exons 2 to 4 of HLA-A*02:01:01 as the ligand domain. In parallel batches, two variants of the CLR were encoded by the lentiviral vector, namely one having a short spacer of 12 AA (SEQ ID NO: 8), and one having a long spacer of 229 AA (SEQ ID NO: 9) of the hinge region of IgG4-Fc. On day 9 after stimulation, the transduced T-cells and K562 control cells were sorted by flow cytometry (FACS) using the co-expressed EGFRt as a selection marker.

Cells expressing the selection marker were cultivated for a further 5 d and analysed for expression of the CLR and for their activity against immune cells directed against the ligand domain of the CLR. The transduced cells were contacted with the supernatant of HB-82 cells containing the BB7.2 antibody. Secondary staining was done by anti-mouse IgG (F(ab)2), labelled with Phycoerythrin. The FACS results are depicted in FIG. 7, showing that the BB7.2 antibody effectively binds to the immune cells expressing the CLR, specifically binding to the ligand domain of the CLR.

FIG. 7 shows FACS results, in A) for control cells, and in B) for T-cells, namely for untransduced T-cells (untransduced T cells) and untransduced control cells (untransduced K562), for cells transduced with the construct containing the short spacer T-cells (HLA I CLR_short T cells) or control cells (HLA I CLR short K562), and for cells transduced with the construct containing the long spacer T-cells (HLA I CLR long T cells) or control cells (HLA I CLR long K562). The results show that the CLR is strongly expressed both in control cells (K562), and in T-cells, wherein the short spacer showed a stronger expression of the CLR, in particular in T cells.

The activity of T-cells expressing the CLR was analysed by contacting and co-cultivating them with freshly washed mouse hybridoma B lymphocyte HB-82, which express the HLA-A2 specific BCR BB7.2, which correlates to the secreted antibody BB7.2. The cells expressing the CLR were used at a ratio of 0.5:1 or 1:1, or 5:1 with HB-82 cells. For all ratios, an effective killing of the HB-82 cells was observed, showing that the expression of the CLR by T-cells results in effective elimination of the B-cells that express a BCR specific for the ligand domain of the CLR. For the CLR having the short spacer, a higher cytotoxic effectivity of the T-cell expressing it was determined. The results of the analysis of lactate dehydrogenase (LDH) released after 48 h of co-cultivation is depicted in FIG. 8 showing that in all ratios for T-cells expressing the CLR an effective cytotoxicity was found.

In addition, the expression of activation markers on T-cells expressing the CLR was analysed after 48 h of co-cultivation at effector-to-target ratios of 5:1 or 1:1, and 0.5:1. The results for CD8+ T-cells are shown in FIG. 9 A) for CD137, in FIG. 9B) for CD69, and in FIG. 9C) for CD25. These results show that the CD8+ T-cells that were transduced to express the CLR clearly showed expression of the activation markers CD137, CD69, and CD25, for all ratios and both for the short and for the long spacer variants, with a stronger activation for the short spacer variant.

These results on the example of a B-cell receptor show that the signalling molecule CLR of the invention directs T-cells that express this signalling molecule to kill immune cells, exemplified by B-cells, with specificity for the cognate antigen against which the immune cells are directed, e.g. with specificity for the B-cell receptor, resp. T-cell receptor, of the immune cells.

1. Fusion protein for use as a signalling molecule, comprising a ligand domain, a spacer, a transmembrane domain, and an intracellular signalling domain, wherein the ligand domain contains at least one epitope of a cognate antigen, against which an undesired immune response is directed.

2. Fusion protein according to claim 1, comprising a dimerization domain arranged between the ligand domain and the transmembrane domain.

3. Fusion protein according to claim 1, wherein the ligand domain comprises two chains, each of the chains of the ligand domain are covalently linked to a dimerization domain, and the dimerization domains are dimerized.

4. Fusion protein according to claim 1, wherein the ligand domain comprises two chains, each of which are linked to a dimerization domain, of which only one is covalently linked to a spacer, a transmembrane domain, and to an intracellular signalling domain.

5. Fusion protein according to claim 1, wherein the ligand domain comprises two chains and each of these chains is covalently linked to a dimerization domain, a spacer, a transmembrane domain, and an intracellular signalling domain, wherein the spacers, transmembrane domains and intracellular signal domains covalently linked to each chain can have the same amino acid sequence or a different amino acid sequence each.

6. Fusion protein according to claim 1, wherein the spacer has a length of 10 to 250 amino acids.

7. Fusion protein according to claim 1, wherein the dimerization domains only dimerize to heterodimers.

8. Fusion protein according to claim 1, wherein the intracellular signalling domain is a combination of a h4-1BB domain and a hCD3ξ domain, or a combination of an intracellular hCD28 signalling domain and a hCD3ξ domain.

9. Fusion protein according to claim 1, wherein the ligand domain comprises at least a portion of a HLA class I or at least a portion of a HLA class II molecule.

10. Fusion protein according to claim 5, wherein the portion of the HLA class I contains at least one mutation in amino acid position No. 74, 223, 224, 225, 226, 227, 229, and 245, wherein the numbering refers to HLA class I without signal peptide.

11. Fusion protein according to claim 5, wherein the portion of the HLA class II contains at least one mutation in amino acid position No. 88, 90 and 176 in the alpha chain, and/or a mutation in at least one amino acid position ofNos. 46, 54, 55, 56, 104, 114, 116, 134, 135, 136, 137, 138, 139, 141, 142, 143, 144, 145, 148, 158, 160, and 162 in the beta chain, wherein the numbering refers to HLA class II without signal peptide.

12. Fusion protein according to claim 1, comprising a first signalling molecule comprising a ligand domain, optionally an extracellular part of a HLA transmembrane domain, a linker, a dimerization domain, a spacer, a transmembrane domain, and at least one intracellular signalling domain, wherein the dimerization domain is dimerized with the dimerization domain of a second signalling molecule, the second signalling molecule comprising a ligand domain, an extracellular part of a HLA transmembrane domain, a linker, a dimerization domain, a spacer, a transmembrane domain, and at least one intracellular signalling domain, or the second signalling molecule consisting of a ligand domain, a linker and a dimerization domain.

13. Fusion protein according to claim 1, comprising a first signalling molecule comprising or consisting of a ligand domain, optionally an extracellular part of a HLA transmembrane domain, a spacer, a transmembrane domain, and at least one intracellular signalling domain.

14. Fusion protein according to claim 1, for use in the treatment of immune rejections against transplants, for use in the treatment of autoimmune diseases, or for use in the treatment of allergies.

15. Fusion protein according to claim 10 for use in the treatment of autoimmune diseases, wherein the ligand domain is the cognate antigen against which the autoimmune disease is directed.

16. Fusion protein according to claim 1, consisting of a first signalling molecule and a second signalling molecule comprising a ligand domain consisting of two chains, wherein the first signalling molecule from N-terminus to C-terminus consists of one chain of the ligand domain, a dimerization domain, a spacer, a transmembrane domain, and an intracellular signalling domain,
wherein the second signalling molecule comprises the other one chain of the ligand domain and a dimerization domain,
wherein the dimerization domain of the first signalling molecule is dimerized with the dimerization domain of the second signalling molecule, and
wherein the ligand domain contains at least one epitope of a cognate antigen, against which an undesired immune response is directed.

17. Fusion protein according to claim 1, wherein the second signalling molecule from N-terminus to C-terminus consists of the other one chain of the ligand domain, a linker and a dimerization domain.

18. Fusion protein according to claim 1, wherein the second signalling molecule from N-terminus to C-terminus consists of the other one chain of the signalling domain, a linker, a dimerization domain, a spacer, a transmembrane domain and an intracellular signalling domain.

19. Fusion protein according to claim 1, wherein the ligand domain is a HLA class II molecule, wherein the one chain of the ligand domain of the first signalling molecule consists of the alpha 1 and optionally alpha 2 domains of the HLA class II molecule, and the other one chain of the ligand domain of the second signalling molecule consists of the beta 1 and optionally beta 2 domains of the HLA class II molecule.

20. Fusion protein according to claim 1, wherein the ligand domain is a HLA class II molecule, wherein the one chain of the ligand domain of the first signalling molecule consists of the beta 1 and optionally beta 2 domains of the HLA class II molecule, and the other one chain of the ligand domain of the second signalling molecule consists of the alpha 1 and optionally alpha 2 domains of the HLA class II molecule.

21. Fusion protein according to claim 1, wherein the ligand domain is a HLA class I molecule and the one chain of of the first signalling molecule is at least a portion of the heavy chain of the HLA class I molecule, and the other one chain of the ligand domain of the second signalling molecule consists of β2-microglobulin having the dimerization domain at its N-terminus and/or at its C-terminus.

22. Immune cell expressing a fusion protein according to claim 1 for use in the treatment of immune rejections against transplants, for use in the treatment of autoimmune diseases, or for use in the treatment of allergies.

23. Immune cell according to claim 22, wherein the immune cell is a T-cell, a primary T-cell, a NK cell, or a progenitor cell of one of these or a cell line.

24. Immune cell according to claim 22, wherein the immune cell is immunologically compatible with the recipient.

25. Immune cell according to claim 1, wherein the immune cell is for administration to a patient prior to or following transplantation.

26. Nucleic acid sequence encoding a fusion protein according to claim 1.


SEQUENCE LISTING (21 sequences)

SEQ ID NO: 1 - protein, 298 aa, Artificial Sequence
  CHAIN 1..24: signal peptide
  antigen domain HLA-A*0201
  CHAIN 25..114: alpha 1 domain
  CHAIN 115..206: alpha 2 domain
  CHAIN 207..298: alpha 3 domain
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRW

SEQ ID NO: 2 - protein, 257 aa, Artificial Sequence
  CHAIN 1..25: signal peptide
  fusion protein
  CHAIN 26..109: alpha 1 domain
  CHAIN 110..203: alpha 2 domain
  CHAIN 204..216: extracellular portion of transmembrane domain
  CHAIN 217..223: linker
  CHAIN 224..257: Jun zipper
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTESRGGGGGARLEEKVKTLKAQNSELASTANMLREQVAQLKQS

SEQ ID NO: 3 - protein, 267 aa, Artificial Sequence
  CHAIN 1..29: signal peptide
  fusion protein
  CHAIN 30..123: beta 1 chain
  CHAIN 124..217: beta 2 chain
  CHAIN 218..227: extracellular part of transmembrane domain
  CHAIN 228..234: linker
  CHAIN 235..267: Fos zipper
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKSRGGGGGDTLQAETDQLEDEKSALQTEIANLLKEKEKLEF

SEQ ID NO: 4 - protein, 34 aa, Artificial Sequence
  CHAIN 1..4: linker
  linker domain-acidic zipper domain
  CHAIN 5..34: acidic zipper
SRGTAQLEKELQALEKENAQLEWELQALEKELAQ

SEQ ID NO: 5 - protein, 34 aa, Artificial Sequence
  CHAIN 1..4: linker
  linker domain-basic zipper domain
  CHAIN 5..34: basic zipper
SRGTAQLKKKLQALKKKNAQLKWKLQALKKKLAQ

SEQ ID NO: 6 - protein, 47 aa, Artificial Sequence
  CHAIN 1..4: linker
  linker domain-REL zipper domain
  CHAIN 5..47: REL zipper
SRGSLEIRAAFLRQRNTALRTEVAELEQEVQRLENEVSQYETRYGPL

SEQ ID NO: 7 - protein, 47 aa, Artificial Sequence
  CHAIN 1..4: linker
  linker domain-ERL zipper domain
  CHAIN 5..47: ERL zipper
SRGTLEIEAAFLERENTALETRVAELRQRVQRLRNRVSQYRTRYGPL

SEQ ID NO: 8 - protein, 873 aa, Artificial Sequence
  CHAIN 1..24: signal peptide
  CLR with short spacer
  CHAIN 25..114: alpha 1 domain
  CHAIN 115..206: alpha 2 domain
  CHAIN 207..298: alpha 3 domain
  CHAIN 299..310: short spacer domain
  CHAIN 311..338: CD28 transmembrane domain
  CHAIN 339..380: 4-1BB costimulatory domain
  CHAIN 381..492: CD3 zeta signalling domain
  CHAIN 493..516: T2A element
  CHAIN 517..538: GM-CSF signal peptide
  CHAIN 539..873: truncated EGFR selection domain
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 9 - protein, 1089 aa, Artificial Sequence
  CHAIN 1..24: signal peptide
  CLR long spacer construct
  CHAIN 25..114: alpha 1 domain
  CHAIN 115..206: alpha 2 domain
  CHAIN 207..298: alpha 3 domain
  CHAIN 299..526: long spacer domain
  CHAIN 527..554: CD28 transmembrane domain
  CHAIN 555..596: 4-1BB costimulatory domain
  CHAIN 597..708: CD3 zeta signalling domain
  CHAIN 709..732: T2A element
  CHAIN 734..754: GM-CSF signal peptide
  CHAIN 755..1089: truncated EGFR selection domain
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 10 - protein, 832 aa, Artificial Sequence
  CHAIN 1..25: signal peptide
  HLA-DRA*0101 CLR construct with Jun-zipper
  CHAIN 26..109: alpha 1 domain
  CHAIN 110..203: alpha 2 domain
  CHAIN 204..216: extracellular part of transmembrane domain
  CHAIN 217..223: linker
  CHAIN 224..257: Jun-Zipper
  CHAIN 258..269: short spacer domain
  CHAIN 270..297: CD28 transmembrane domain
  CHAIN 298..339: 4-1BB costimulatory domain
  CHAIN 340..451: CD32 zeta signalling domain
  CHAIN 452..475: T2A element
  CHAIN 476..497: GM-CSF signal peptide
  CHAIN 498..832: truncated EGFR selection domain
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTESRGGGGGARLEEKVKTLKAQNSELASTANMLREQVAQLKQSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 11 - protein, 842 aa, Artificial Sequence
  CHAIN 1..29: signal peptide
  HLA-DRB1*0101 CLR short construct with Fos- zipper
  CHAIN 30..123: beta 1 domain
  CHAIN 124..217: beta 2 domain
  CHAIN 218..227: extracellular part of transmembrane domain
  CHAIN 228..234: linker
  CHAIN 235..267: Fos-Zipper
  CHAIN 268..279: short hinge domain short spacer domain
  CHAIN 280..307: CD28 transmembrane domain
  CHAIN 308..349: 4-1BB costimulatory domain
  CHAIN 350..461: CD3 zeta signalling domain
  CHAIN 462..485: T2A element
  CHAIN 486..507: GM-CSF signal peptide
  CHAIN 508..842: truncated EGFR selection domain
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKSRGGGGGDTLQAETDQLEDEKSALQTEIANLLKEKEKLEFESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 12 - protein, 1048 aa, Artificial Sequence
  CHAIN 1..25: signal peptide
  HLA-DRA*0101 CLR long construct with Jun- Zipper
  CHAIN 26..109: alpha 1 domain
  CHAIN 110..203: alpha 2 domain
  CHAIN 204..216: extracellular part of transmembrane domain
  CHAIN 217..223: linker
  CHAIN 224..257: Jun-Zipper
  CHAIN 258..485: long spacer domain
  CHAIN 486..513: CD28 transmembrane domain
  CHAIN 514..555: 4-1BB costimulatory domain
  CHAIN 556..667: CD3 zeta signalling domain
  CHAIN 668..691: T2A element
  CHAIN 692..713: GM-CSF signal peptide
  CHAIN 714..1048: truncated EGFR selection domain
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTESRGGGGGARLEEKVKTLKAQNSELASTANMLREQVAQLKQSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 13 - protein, 1058 aa, Artificial Sequence
  CHAIN 1..29: signal peptide
  HLA-DRB1*0101 CLR long construct with Fos- Zipper
  CHAIN 30..123: beta 1 domain
  CHAIN 124..217: beta 2 domain
  CHAIN 218..227: extracellular part of transmembrane domain
  CHAIN 228..234: linker
  CHAIN 234..267: Fos-Zipper
  CHAIN 268..495: long spacer domain
  CHAIN 496..523: CD28 transmembrane domain
  CHAIN 524..565: 4-1BB costimulatory domain
  CHAIN 566..677: CD3 zeta signalling domain
  CHAIN 678..701: T2A element
  CHAIN 702..723: GM-CSF signal peptide
  CHAIN 724..1058: truncated EGFR selection domain
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKSRGGGGGDTLQAETDQLEDEKSALQTEIANLLKEKEKLEFESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 14 - protein, 832 aa, Artificial Sequence; identical to SEQ ID NO: 10
  CHAIN 1..25: signal peptide
  HLA-DRA*0101 CLR short construct with Jun- Zipper
  CHAIN 26..109: alpha 1 chain
  CHAIN 110..203: alpha 2 chain
  CHAIN 204..216: extrcellular part of transmembrane domain
  CHAIN 217..223: linker
  CHAIN 224..257: Jun-Zipper
  CHAIN 258..269: short spacer domain
  CHAIN 270..297: CD28 transmembrane domain
  CHAIN 298..339: 4-1BB costimulatory domain
  CHAIN 340..451: CD3 zeta signalling domain
  CHAIN 452..475: T2A element
  CHAIN 476..497: GM-CSF signal peptide
  CHAIN 498..832: truncated EGFR selection domain

SEQ ID NO: 15 - protein, 267 aa, Artificial Sequence; identical to SEQ ID NO: 3
  CHAIN 1..29: signal peptide
  HLA-DRB1*0101 ligand with linker and Fos- Zipper
  CHAIN 30..123: beta 1 domain
  CHAIN 124..217: beta 2 domain
  CHAIN 218..227: extracellular part of transmembrane domain
  CHAIN 228..234: linker
  CHAIN 235..267: Fos-Zipper

SEQ ID NO: 16 - protein, 1048 aa, Artificial Sequence; identical to SEQ ID NO: 12
  CHAIN 1..25: signal peptide
  HLA-DRA*0101 CLR long construct with Jun- Zipper
  CHAIN 26..109: alpha 1 domain
  CHAIN 110..203: alpha 2 domain
  CHAIN 204..216: extracellular part of transmembrane domain
  CHAIN 217..223: linker
  CHAIN 224..257: Jun-Zipper
  CHAIN 258..485: long spacer domain
  CHAIN 486..513: CD28 transmembrane domain
  CHAIN 514..555: 4-1BB costimulatory domain
  CHAIN 556..667: CD3 zeta signalling domain
  CHAIN 668..691: T2A element
  CHAIN 692..713: GM-CSF signal peptied
  CHAIN 714..1048: EGFR selection domain

SEQ ID NO: 17 - protein, 267 aa, Artificial Sequence; identical to SEQ ID NO: 3
  CHAIN 1..29: signal peptide
  HLA-DRB1*0101 ligand with linker and Fos- Zipper
  CHAIN 30..123: beta 1 domain
  CHAIN 124..217: beta 2 domain
  CHAIN 218..227: extracellular part of transmembrane domain
  CHAIN 228..234: linker
  CHAIN 235..267: Fos-Zipper

SEQ ID NO: 18 - protein, 842 aa, Artificial Sequence; identical to SEQ ID NO: 11
  CHAIN 1..29: signal peptide
  HLA-DRB1*0101 CLR short construct with Fos- Zipper
  CHAIN 30..123: beta 1 domain
  CHAIN 124..217: beta 2 domain
  CHAIN 218..227: extracellular part of transmembrane domain
  CHAIN 228..234: linker
  CHAIN 235..267: Fos-Zipper
  CHAIN 268..279: short spacer domain
  CHAIN 280..307: CD28 transmembrane domain
  CHAIN 308..349: 4-1BB costimulatory domain
  CHAIN 350..461: CD3 zeta signalling domain
  CHAIN 462..485: T2A element
  CHAIN 486..507: GM-CSF signal peptide
  CHAIN 508..842: truncated EGFR selection domain

SEQ ID NO: 19 - protein, 257 aa, Artificial Sequence; identical to SEQ ID NO: 2
  CHAIN 1..25: signal peptide
  HLA-DRA*0101 ligand with linker and Jun-Zipper
  CHAIN 26..109: alpha 1 chain
  CHAIN 110..203: alpha 2 chain
  CHAIN 204..216: extracellular part of transmembrane domain
  CHAIN 217..223: linker
  CHAIN 224..257: Jun-Zipper

SEQ ID NO: 20 - protein, 1058 aa, Artificial Sequence; identical to SEQ ID NO: 13
  CHAIN 1..29: signal peptide
  HLA-DRB1*0101 CLR long construct with Fos- Zipper
  CHAIN 30..123: beta 1 domain
  CHAIN 124..217: beta 2 domain
  CHAIN 218..227: extracellular part of transmembrane domain
  CHAIN 228..234: linker
  CHAIN 235..267: Fos-Zipper
  CHAIN 268..495: long spacer domain
  CHAIN 496..523: CD28 transmembrane domain
  CHAIN 524..565: 4-1BB costimulatory domain
  CHAIN 566..677: CD3 zeta signalling domain
  CHAIN 678..701: T2A element
  CHAIN 702..723: GM-CSF signal peptide
  CHAIN 724..1058: EGFR selection domain

SEQ ID NO: 21 - protein, 257 aa, Artificial Sequence; identical to SEQ ID NO: 2
  CHAIN 1..25: signal peptide
  HLA-DRA*0101 Ligand with linker and Jun-Zipper
  CHAIN 26..109: alpha 1 domain
  CHAIN 110..203: alpha 2 domain
  CHAIN 204..216: extracellular part of transmembrane domain
  CHAIN 217..223: linker
  CHAIN 224..257: Jun-Zipper